(19) United States
(12) Patent Application Publication
(10) Pub. No.: US 2014/0271954 A1
(43) Pub. Date: Sep. 18, 2014

(54) Toxin Expelling Powder Comprising Root of Buckwheat and Preparation Process Thereof

(71) Applicants: Junhong Zhao, Chongqing (CN); Youqian Zhao, Chongqing (CN)

(72) Inventors: Junhong Zhao, Chongqing (CN); Youqian Zhao, Chongqing (CN)

(21) Appl. No.: 14/353,714

(22) PCT Filed: Jun. 28, 2013
(86) PCT No.: PCT/CN2013/000780

§ 371 Date: Apr. 23, 2014

(30) Foreign Application Priority Data
Sep. 13, 2012 (CN) 201210337010.5

(51) Int. Cl. A61K 36/70; A61K 36/489; A61K 36/42; A61K 36/9068

(52) U.S. Cl. CPC A61K 36/70; A61K 36/9068; A61K 36/489; A61K 36/42
USPC 424/756

(57) ABSTRACT

The present invention provides tartary buckwheat detox powder and a method for preparing the same. Raw materials are loveyly hemsleya root, Sophora flavescens root, tartary buckwheat and dried ginger. The method comprises precessing the materials by freezing, wetting, acidifying, alkalizing, salifying, fermenting, steaming, boiling, baking, etc.; crushing and then formulating the resultant into watered pills or capsules, packaging and storing. The detox powder can be used for treating fatty liver disease, alcohol liver disease, hepatitis, liver cancer, liver cirrhosis, and ascites accompanied with liver cancer of late stage.

### BACKGROUND

1. Field of the Invention

The invention relates to the technical field of preparing medicinal materials, in particular to tartary buckwheat detox powder, its unique formulation and a preparation method therefor.

2. Background

Liver disease is a type of common and extremely harmful diseases. According to incomplete statistics, in China there are more than 100 million people infected with hepatitis B virus and 20 million people suffer from chronic hepatitis B disease. Thus, China is worthy of the name of a big-hepatitis B-disease country. In China, hepatitis B disease accounts for more than 80% of existing cases with serious diseases such as liver cirrhosis and liver cancer. As can be seen, the medicine will have a wide development prospect if its effectiveness is verified in studies.

### SUMMARY OF THE INVENTION

The present invention provides tartary buckwheat detox powder for soothing liver and a method for preparing the same. The tartary buckwheat detox powder has good effects for treating fatty liver disease, alcohol liver disease, hepatitis, liver cirrhosis, liver cancer, and ascites accompanied with liver cirrhosis and liver cancer of late stage, and thus presents a promising market prospect and a wide development potential. Moreover, the material resource for preparing the medicine is abundant, the technical operation for preparing the medicine is simply, and the necessary production investment is low.

One object of the present invention is to provide tartary buckwheat detox powder. The tartary buckwheat detox powder consists of loveyly hemsleya root, Sophora flavescens root, tartary buckwheat and dried ginger as main ingredients, with weight percentage as follows: 10 to 35% of loveyly hemsleya root, 10 to 35% of Sophora flavescens root, 15 to 25% of tartary buckwheat and 15 to 25% of dried ginger.

Another object of the present invention is to provide a method for preparing the tartary buckwheat detox powder, the method comprises the following steps:

Step 1, screening out qualified Chinese medicine decoction pieces and qualified taratry buckwheat as raw materials;

Step 2, processing the decoction pieces and the tartary buckwheat by freezing, wetting, acidifying, alkalizing, salifying, fermenting, steaming, boiling, baking, etc;

Step 3, crushing, and then formulating the resultant into watered pill or capsule, packaging and storing.

Further, the loveyly hemsleya root and Sophora flavescens root used originates from Aba Tibetan Autonomous Prefecture of Sichuan province, the tartary buckwheat used from Yunnan, Guizhou or Sichuan province, and the dried ginger used is Curcuma longa L.

The present invention provides tartary buckwheat detox powder and a method for preparing the same. The main ingredients of the tartary buckwheat detox powder are Loveyly hemsleya root, Sophora flavescens root, tartary buckwheat and dried ginger. The pharmacological principle of the tartary buckwheat detox powder is to rapidly detoxifying liver, and the tartary buckwheat detox powder specifically treats fatty liver disease, alcohol liver disease, hepatitis, liver cirrhosis, liver cancer, ascites accompanied with liver cirrhosis and liver cancer of late stage. In preparation of the tartary buckwheat detox powder, the qualified Chinese medicine decoction pieces and the qualified tartary buckwheat are firstly processed by freezing, wetting, acidifying, alkalizing, salifying, fermenting, steaming, boiling, baking, etc.; the resultant, after being crushedis formulated into watered pills or capsules, packaged and stored. The medicine has a promising market prospect and a wide development potential; moreover, its material resource is abundant, its preparation operation is simple, ant its production investment is low, all of which render the medicine applicable and propagable. A prescription modulation thereof for treating constipation presents special curative effects, i.e., radical cure can be achieved after successively taking the medicine over ten days. Another prescription modulation thereof for treating toothache also presents special curative effects, i.e., recovery can be rapidly achieved within several hours, and loosed teeth will automatically reset and anchor.

### BRIEF DESCRIPTION OF THE FIGURES

FIG. 1 is a flow chart for implementing the method for preparing the tartary buckwheat detox powder according to embodiments of the present invention.

### DETAILED DESCRIPTION OF THE SEVERAL EMBODIMENTS

To make the objects, technical solutions and advantages of the present invention more apparent, the present invention will be further described in combination with the accompanying figure and embodiments. It should be understood that the specific embodiments described herein are merely examples to illustrate the invention, not to limit the present invention.

The object of the present invention is to provide tartary buckwheat detox powder. The main ingredients of the tartary buckwheat detox powder are loveyly hemsleya root, Sophora flavescens root, tartary buckwheat, and dried ginger.

In embodiments of the present invention, the pharmacological principle of the tartary buckwheat detox powder is to rapidly detoxifying liver, and therefore it can be used to treat fatty liver disease, alcohol liver disease, hepatitis, liver cirrhosis, liver cancer, and ascites accompanied with liver cirrhosis and liver cancer of late stage.

FIG. 1 shows a flow for implementing the method for preparing the tartary buckwheat detox powder according to embodiments of the present invention. The method comprises the following steps:

Step S101, screening out qualified Chinese medicine decoction pieces and qualified taratry buckwheat as raw materials;

Step 102, processing the qualified decoction pieces and the qualified tartary buckwheat by freezing, wetting, acidifying, alkalizing, salifying, fermenting, steaming, boiling, baking, etc;

Step 103, crushing and then formulating the resultant into watered pill or capsule, packaging and storing.

Application principles of the present invention will be further described herein in combination with the accompanying figure and specific embodiments.

The tartary buckwheat detox powder is prepared from such Chinese medicine as loveyly hemsleya root, Sophora flavescens root, tartary buckwheat, and dried ginger, and has functions of clearing heat, detoxifying, and promoting bowel movement. Clinical applications present good effects for treating fatty liver disease, alcohol liver disease, hepatitis B, liver cirrhosis, liver ascites, liver cancer, etc.

The medicine has been used for nearly ten years and cured thousands of patients sufferring from liver diseases. Feedbacks from those who took the medicine indicates that the medicine works very well. For patients who have taken the medicine for one month, the transaminase index can be controlled, and the situation that HbsAg, HbeAg and HbcAb test is positive can be progressively transferred to the situation that HBsAg, HBeAb and HBcAb test is positive and recovery. The symptoms of patients were remarkably improved. For a patient with liver cirrhosis of late stage who insisted in taking the medicine, the conditions were well controlled, liver cirrhosis did not worsen, and some indicators tested negative. As seen from feedbacks, the tartary buckwheat detox powder presents functions of detoxifying, enhancing immunity, and restoring liver function. Despite its cold and cool property, it bears no toxic and side effects.

The embodiments of the present invention provide the tartary buckwheat detox powder and a method for preparing the same. The main ingredients of the tartary buckwheat detox powder are loveyly hemsleya root, Sophora flavescens root, tartary buckwheat, and dried ginger. The pharmacological principle of the tartary buckwheat detox powder is to rapidly detoxifying liver. The tartary buckwheat detox powder specifically treats fatty liver disease, alcohol liver disease, hepatitis, liver cirrhosis, liver cancer, and ascites accompanied with liver cirrhosis and liver cancer of late stage. In preparation of the tartary buckwheat detox powder, the qualified Chinese medicine decoction pieces and the qualified tartary buckwheat are firstly processed by freezing, wetting, acidifying, alkalizing, salifying, fermenting, steaming, boiling, baking, etc.; the resultant, after being crushed, is formulated into watered pills or capsules packaged and stored. The medicine has a promising market prospect and a wide development potential; moreover, its material resource is abundant, its preparation operation is simple, ant its production investment is low, all of which render the medicine applicable and propagable.

The foregoing are preferred embodiments of the present invention only and not intended to limit the present invention. Any modification, equivalent alteration and modification, etc., made within the spirit and principle of the present invention, should be encompassed in the protection scope of the present invention.

What is claimed is:

1. Tartary buckwheat detox powder, characterized in that, the tartary buckwheat detox powder comprises loveyly hemsleya root, Sophora flavescens root, tartary buckwheat, dried ginger as main ingredients with their weight percentages as follows: 10 to 35% of loveyly hemsleya root, 10 to 35% of Sophora flavescens root, 15 to 25% of tartary buckwheat and 15 to 25% of dried giner.

2. A method for preparing the tartary buckwheat detox powder according to claim 1, characterized in that, the method comprises the following steps:
Step 1, screening out qualified Chinese medicine decoction pieces and qualified taratry buckwheat as raw materials;
Step 2, processing the Chinese medicine decoction pieces and the tartary buckwheat by freezing, wetting, acidifying, alkalizing, salifying, fermenting, steaming, boiling, baking, etc;
Step 3, crushing and then formulating the resultant into watered pill or capsule, packaging and storing.

3. The method according to claim 2, characterized in that, loveyly hemsleya root and Sophora flavescens root used originate from Aba Tibetan Autonomous Prefecture of Sichuan province, the tartary buckwheat used originates from Yunnan, Guizhou or Sichuan province, the dried ginger used is Curcuma longa L.
